(19) United States
(12) Patent Application Publication
(10) Pub. No.: US 2026/0301903 A1
(43) Pub. Date: Oct. 1, 2026

(54) SYSTEM AND METHOD FOR GENERATING RANDOMIZED DOSING INSTRUCTIONS FOR DRUG ADMINISTRATION

(71) Applicant: James William Shaughnessy, Port Washington, NY (US)

(72) Inventor: James William Shaughnessy, Port Washington, NY (US)

(21) Appl. No.: 19/096,726

(22) Filed: Apr. 1, 2025

(51) Int. Cl. G16H 20/10; G16H 10/60; G16H 80/00

(52) U.S. Cl. CPC G16H 20/10; G16H 10/60; G16H 80/00

(57) ABSTRACT

Systems and methods for generating randomized dosing instructions for drug administration is disclosed. The system comprises a processor and memory configured to receive user-defined parameters including minimum and maximum dosage, permissible administration routes, and intervals between doses. A randomization engine utilizing a secondary entropy source generates clinically safe but unpredictable timing intervals, dose amounts, and ingestion methods. Generated instructions are compiled and transmitted electronically to patients through selectable notification channels. The invention enables automated tapering through dynamic adjustment of dosing parameters without user perception of systematic reductions. By eliminating predictable dosing intervals and leveraging computational randomness bounded within safe therapeutic parameters, the system effectively mitigates psychological habituation and anticipatory behavior, enhancing medication adherence and therapeutic efficacy. Alternative embodiments extend the invention's applicability to chronic pain management, nutritional supplements, psychiatric treatments, and clinical research protocols requiring controlled, randomized dosage schedules.

### TECHNICAL FIELD

The present disclosure relates generally to the field of drug administration systems. More specifically, it pertains to systems and methods for controlling drug dosage and timing in a configurable and adaptive manner.

### BACKGROUND OF THE INVENTION

Drug addiction, medication overuse, and poor adherence to prescribed dosing regimens represent enduring challenges in both clinical and non-clinical healthcare environments. Despite numerous advancements in pharmaceutical formulations and behavioral therapies, achieving controlled, sustainable, and patient-compliant drug use remains an elusive goal in many treatment programs. Particularly in cases involving narcotic or habit-forming substances, the process of tapering off a drug must strike a difficult balance between effectiveness, patient safety, and user engagement.

In conventional medicine management systems, dosing regimens are typically fixed or scheduled according to a predictable timeline. Patients are instructed to take a specified quantity of medication at regular intervals—e.g., every 4, 6, or 12 hours. These fixed schedules are useful in the context of acute care or chronic condition management where maintaining a steady concentration of a drug in the bloodstream is desirable. However, in the context of behavioral health, addiction treatment, and even certain chronic pain regimens, these rigid structures may lead to adverse outcomes, including psychological dependence on the schedule itself.

One of the key problems with fixed dosing intervals is the emergence of anticipatory behavior. Patients often begin to fixate on the next upcoming dose, developing heightened cravings or anxiety in the hours leading up to it. This behavioral pattern has been extensively documented in the literature on substance use and is understood to increase the risk of relapse. The psychological association of relief with the time of dose intake can itself become a reinforcing mechanism. In turn, this can undermine the intended tapering or recovery process.

To mitigate these concerns, clinicians sometimes employ “as-needed” (PRN) prescriptions that give patients the discretion to decide when to take a medication within general bounds. While this introduces flexibility, it also introduces significant risk, particularly with substances that carry a high potential for abuse. Without strict oversight or monitoring, PRN prescriptions may enable misuse, overuse, or erratic dosing behavior.

Other attempts to address the same concern have included physical medication dispensers or pill boxes equipped with alarms and locks. These systems enforce time-based dosing and sometimes include digital tracking or require biometric access. While helpful in improving adherence and limiting unauthorized access, they generally still operate on predictable timing intervals. Patients quickly learn the pattern of availability, allowing the same anticipatory behavior to develop. Furthermore, such hardware-based solutions may be cost-prohibitive, non-adaptive, or lack the flexibility to support evolving treatment goals such as gradual dose tapering.

In digital health settings, mobile and web-based applications have emerged to support medication adherence through reminders, calendars, and interactive check-ins. These systems often include journaling features, progress tracking, and even direct communication with healthcare providers. However, most of these tools reinforce schedule-based routines. While some may allow minor variations in timing, they do not substantially deviate from the core paradigm of structured, repeated schedules. As such, they fail to disrupt the behavioral predictability that contributes to compulsive medication seeking or psychological dependence.

In the domain of addiction treatment specifically, medication-assisted treatment (MAT) programs employ structured dosing of drugs like buprenorphine or methadone to reduce withdrawal symptoms and curb cravings. While these programs are evidence-based and effective, they typically involve regular in-person administration or tightly controlled take-home protocols. These methods limit the possibility for randomization or dynamic adjustment based on individual behavior. Moreover, they may not scale well or accommodate patients who do not engage well with rigid institutional structures.

Another common intervention is the use of digital timers and alarms paired with behavioral therapy. Cognitive-behavioral therapy (CBT) often includes exercises to delay gratification and stretch intervals between doses. However, these approaches depend heavily on patient willpower and may be undermined by the very predictability of their dosing environments. Where software has been used to assist, it typically functions in a deterministic manner, generating suggestions or reminders according to pre-set logic rather than responsive or randomized inputs.

Attempts to integrate AI or data-driven insights into dosing routines are still in early stages and often focus on personalized dosage optimization or adverse event prediction. These systems rely on large datasets and clinical oversight, limiting their accessibility or adaptability in outpatient or self-administered contexts. Moreover, these systems are often not designed to accommodate the needs of patients in recovery or tapering processes, and rarely explore the behavioral implications of timing and delivery method variation.

Another technical limitation of existing systems is that dose adjustments or tapering protocols typically involve explicit step-down schedules: reducing dose by a fixed percentage every week, for example. While mathematically sound, such step-down methods are transparent to the patient and thus may create anxiety or resistance. Patients may become aware of the decreasing trend and respond with increased cravings or non-compliance. Some may even attempt to “hoard” medication or manipulate the system to receive larger doses sooner. The transparency and predictability of such step-down approaches, while beneficial for oversight, may be counterproductive for engagement.

Furthermore, traditional systems rarely incorporate variation in route of administration. While clinicians recognize that methods like injection, oral ingestion, or transdermal patches differ in bioavailability, onset time, and user experience, most medication management tools do not account for this dimension. For patients tapering off drugs, shifting to a less intense or slower absorption method can aid in desensitization and psychological adjustment. However, this variable is typically ignored in conventional scheduling or automated dispensing systems.

In addition to the structural issues noted above, many systems fail to leverage well-documented psychological mechanisms such as the variable reward schedule—a principle drawn from behavioral psychology which suggests that random and unpredictable reinforcement can reduce compulsive behaviors and increase engagement. This principle has been widely applied in fields like gambling, video game design, and habit formation, yet remains largely absent from medication management. Systems that do not engage users in psychologically relevant ways are more likely to lose their attention or fail to change their behavior over time.

Taken together, these deficiencies expose a technical gap: the need for a drug dispensing or instruction system that can dynamically vary the dose, timing, and route of administration in a way that is still controlled within safe bounds, but unpredictable enough to disrupt anticipatory behavior and reduce psychological dependence. Such a system would ideally allow for personalized parameter setting by a clinician or patient, adaptive tapering, and secure notification of dosing instructions—all while maintaining variability in schedule and method. Additionally, it would benefit from logging and audit capabilities for accountability, while still enabling randomization mechanisms that reduce behavioral reinforcement patterns associated with compulsive drug use.

There remains a need for a flexible, automated, and behaviorally-informed system that addresses the shortcomings of both hardware and software-based conventional solutions, particularly for patients attempting to taper usage or transition to safer forms of administration. This system must balance safety, unpredictability, engagement, and adaptability in a manner not achieved by currently available solutions.

### SUMMARY OF THE INVENTION

In light of the disadvantages mentioned in the previous section, the following summary is provided to facilitate an understanding of some of the innovative features unique to the present invention and is not intended to be a full description. A full appreciation of the various aspects of the invention can be gained by taking the entire specification and drawings as a whole.

The present invention provides a system and method for generating randomized dosing instructions for drug administration. The system accepts user-defined parameters including minimum and maximum dosage, minimum and maximum intervals between doses, and a set of permissible routes of administration. Leveraging a random number generator enhanced by a secondary source of entropy, the system selects a random dose, a random time interval, and a random ingestion method, all within the defined constraints. When the designated time has elapsed, the system transmits a dosing instruction to the user, specifying the dose and method of administration. Each instruction is optionally logged with a timestamp, and the system can transmit notifications via various channels such as email or SMS.

In one embodiment, the system also supports tapering protocols by automatically adjusting dose and interval parameters over time. The randomization of dosing intervals and administration routes introduces unpredictability, disrupting behavioral patterns such as anticipatory craving or schedule-based fixation. This unpredictability, controlled within safe therapeutic bounds, harnesses principles from behavioral psychology such as variable reward scheduling to increase engagement and reduce misuse potential. The system can be implemented as software, hardware, or a combination thereof, and may include user interaction features for start/stop control, compliance feedback, or adaptation based on historical usage.

This summary is provided merely for purposes of summarizing some example embodiments, to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following detailed description and figures.

The abovementioned embodiments and further variations of the proposed invention are discussed further in the detailed description.

### BRIEF DESCRIPTION OF THE DRAWINGS

The accompanying drawings, which are incorporated herein and constitute a part of the specification, illustrate embodiments of the present invention and, together with the description, serve to explain the principles of the invention.

FIG. 1 is a block diagram 100 illustrating the architecture of the random drug dispenser system including processor, memory, and various functional units according to the embodiments of the present disclosure.

FIG. 2 is a schematic 200 showing the functional data flow between configuration, randomization, control, and output components within the system according to the embodiments of the present disclosure.

FIG. 3 is a flow diagram 300 of a method for generating randomized dosing instructions according to the embodiments of the present disclosure.

FIG. 4 is an exemplary depiction 400 of a computing system executing instructions stored in a machine-readable medium for generating and transmitting randomized dosing instructions according to the embodiments of the present disclosure.

FIG. 5 is an exemplary illustration 500 of a user interface depicting a practical example of the application and its user interface according to the embodiments of the present disclosure.

The drawings described herein are for illustration purposes only and are not intended to limit the scope of the present subject matter in any way according to the embodiments of the present disclosure.

### DETAILED DESCRIPTION

In the following description of the embodiments of the invention, reference is made to the accompanying drawings that form a part hereof, and in which are shown by way of illustration specific embodiments in which the invention may be practiced. These embodiments are described in sufficient detail to enable those skilled in the art to practice the invention, and it is to be understood that other embodiments maybe utilized and that changes may be made without departing from the scope of the present invention. The following detailed description is, therefore, not to be taken in a limited sense, and the scope of the present invention is defined only by the appended claims.

The specification may refer to “an”, “one” or “some” embodiment(s) in several locations. This does not necessarily imply that each such reference is to the same embodiment(s), or that the feature only applies to a single embodiment. A single feature of different embodiments may also be combined to provide other embodiments.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the foregoing sections, some features are grouped together in a single embodiment for streamlining the disclosure. This method of disclosure is not to be interpreted as reflecting an intention that the disclosed embodiments of the present disclosure must use more features than are expressly recited in each claim. Rather, as the following claims reflect, the inventive subject matter lies in less than all features of a single disclosed embodiment. Thus, the following claims are hereby incorporated into the detailed description, with each claim standing on its own as a separate embodiment.

Effective medication management, particularly for tapering addictive or habit-forming substances, is challenged by the limitations of fixed dosing schedules and deterministic administration routines. Patients often develop psychological dependencies not only on the drug itself but on the timing and predictability of their dose schedules. Such patterns can undermine tapering efforts, increase relapse risk, and reduce treatment adherence, especially in outpatient or self-managed contexts.

The core technical problem lies in the inability of conventional systems to introduce controlled variability into dosing instructions—variability that is both random enough to disrupt behavioral conditioning and bounded enough to ensure safety. Existing systems largely rely on fixed schedules, simple timers, or patient self-reporting. They do not provide mechanisms to dynamically vary dose timing, quantity, and administration route in a way that maintains clinical safety while supporting psychological disengagement from addictive behaviors.

The present invention addresses this gap by introducing a system that generates dosing instructions based on random selection of timing, dosage, and ingestion method. The randomness is derived from a pseudo-random number generator enhanced by a secondary entropy source such as a hardware-based noise generator or cryptographic function. This ensures that dose intervals and values are unpredictable yet reproducible and bounded within safety constraints defined by the user or clinician.

To operate, the system initializes with user-defined constraints for dose amount (minimum, maximum, and dose unit), time interval (minimum and maximum), and permissible administration methods. At runtime, the system waits a randomly generated interval, then selects a dose and a route of administration at random from the permissible options. Once selected, the system generates a dosing instruction and transmits it to the user through one or more notification channels. Each instruction may also be logged to maintain traceability and support retrospective review.

In some embodiments, the system further enables automated tapering by dynamically updating the bounds for dose and interval over time. This allows the average dosage and frequency to decline gradually without requiring the patient to consciously recognize or adhere to a step-down plan. This feature, in conjunction with randomization and route variability, provides a novel behavioral and technical solution to the long-standing problem of medication tapering in a psychologically sensitive, adaptive, and secure manner.

The present invention relates to a system and method for generating randomized dosing instructions for drug administration. The system allows for configurable constraints on dosage amount, timing intervals, and route of administration, and operates by selecting these dosing parameters randomly within user-defined safety bounds. The randomized instruction is then transmitted to the user, such as through a mobile application, email, or text message, and optionally logged for auditing or clinical supervision purposes.

In one embodiment, the system comprises at least one processor and a memory storing program instructions executable by the processor. The memory may store a configuration module, a randomization engine, a dosing instruction engine, a communication module, and a logging module. The configuration module receives user-defined parameters including minimum and maximum dose (for instance, 1 mg to 5 mg), the dose unit (such as increments of 0.5 mg), minimum and maximum time interval between doses (for example, 2 to 6 hours), and a set of permissible ingestion routes. The randomization engine includes a pseudo-random number generator enhanced by a secondary entropy source, such as hardware-based noise sampling or cryptographic entropy, to ensure output unpredictability. This module generates a random interval between the configured minimum and maximum time, a random dose value snapped to the nearest allowable dose unit within the defined dose bounds, and a random selection of an enabled ingestion route. The dosing instruction engine waits for the randomly selected interval to elapse, and upon completion, it compiles a dosing instruction that includes the selected dose and method of administration, for example, “Please take 1.5 mg of aspirin via transdermal patch.” The communication module transmits the dosing instruction to the user through one or more communication channels, such as push notifications, emails, or SMS messages. The logging module records each dosing event to a timestamped status log that includes the time of issuance, dose amount, and route of administration; this log may be reviewed by clinicians or caregivers for oversight or to adjust tapering settings.

The system optionally supports an automatic tapering function in which the configuration module incrementally adjusts the upper bounds of dose and frequency parameters over time. For example, after a given period, it may reduce the maximum allowable dose by a certain amount and increase the minimum time interval, thereby lowering average intake without requiring the patient to consciously follow a step-down plan. This feature, combined with the randomness of instruction delivery, is designed to reduce behavioral reinforcement patterns that commonly arise from predictable or user-perceived diminishing dose schedules. In practice, the randomness disrupts anticipatory behavior associated with scheduled doses, while the safe parameter bounds protect against over-or under-medication.

In one implementation, the system may be embodied in a mobile or web-based software application providing an interface with a start/stop button to initiate or halt the random dosing process, sliders for setting minimum and maximum dose values and time intervals, and toggles to enable or disable specific ingestion methods. Notification settings allow the user to select one or more preferred channels for receiving instructions, and a status log viewer displays a chronological list of previously issued instructions including dose, route, and timestamp. The interface may also show a status indicator for forthcoming doses and can optionally allow for compliance interactions such as marking a dose as taken, snoozing, or skipping. These compliance inputs can inform adaptive logic in the system, enabling it to refine future instructions based on user feedback or behavior.

In operation, a typical usage scenario might involve a patient launching the application, inputting their desired settings for min/max doses, time intervals, and enabled methods, and pressing “Start.” The system would then randomly select a time interval, dose, and route, wait for the interval to elapse, generate a specific instruction, and transmit it. The user would confirm receipt or compliance, and the event would be logged. Over time, the maximum dose could be gradually reduced while the minimum interval is extended, tapering drug use in a less predictable manner. This combination of technical functionality and behavioral strategies addresses the well-known challenges of anticipatory craving, schedule fixation, and user disengagement in medication or substance dependency contexts.

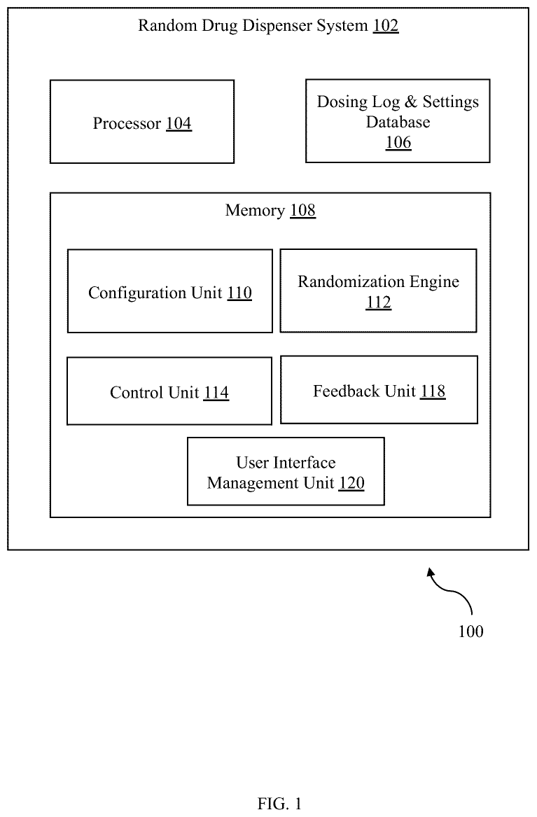

Referring to the figures, FIG. 1 illustrates a block diagram 100 of a Random Drug Dispenser System 102 according to the embodiments of the present disclosure. The Random Drug Dispenser System 102 includes a processor 104, a dosing log and settings database 106, and a memory 108. The dosing log and settings database 106 stores user configurations, historical dosing data, and settings related to permissible dosing parameters. The memory 108 comprises various functional units, including a configuration unit 110, a randomization engine 112, a control unit 114, a feedback unit 118, and a user interface management unit 120. The configuration unit 110 receives and manages user-defined parameters such as minimum and maximum dosages, permissible ingestion routes, and time intervals between doses. The randomization engine 112 generates random dosage amounts, ingestion methods, and timing intervals based on the parameters defined in the configuration unit 110. The control unit 114 manages and coordinates overall system operations, including triggering randomization events, compiling dosing instructions, and handling communication between units. The feedback unit 118 collects and processes user compliance and feedback data, which may be used to dynamically adjust dosing parameters or inform tapering logic. The user interface management unit 120 controls the interactions with the user, including the presentation of dosing instructions, notifications, and the provision of an interface for adjusting settings.

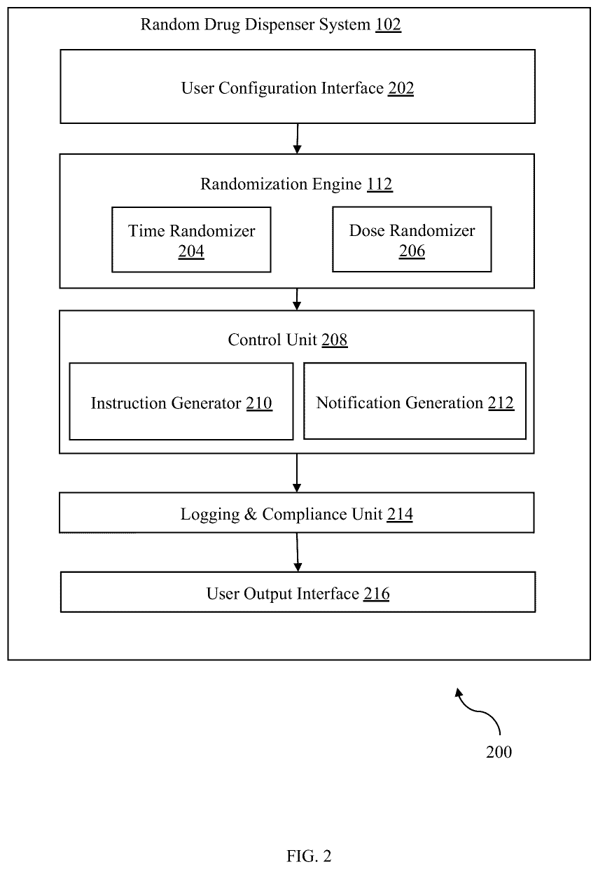

FIG. 2 presents a schematic diagram 200 showing the logical flow of information through the system. The process begins with the user configuration interface 102, where the user (or clinician) defines the minimum and maximum dose ranges, the allowable interval between doses, and which ingestion methods are enabled. These parameters are passed to the randomization module 110, which comprises two components: the time randomizer 110a and the dose and method randomizer 110b. The time randomizer 110a selects a random wait interval between doses using a secure entropy source. Once the interval elapses, the dose and method randomizer 110b selects a random dosage and ingestion method from the set of allowed values. The selections are passed to the control and processing unit 112, which includes an instruction generator 112a that formats the dosing instruction and a notification engine 112b that selects appropriate delivery channels. Before output, the instruction is optionally logged in the logging and compliance module 113 for future review or auditing. Finally, the instruction is sent to the user output interface 114, which may be a mobile application, desktop interface, or another endpoint, where the user receives the dosing instruction and may interact with it.

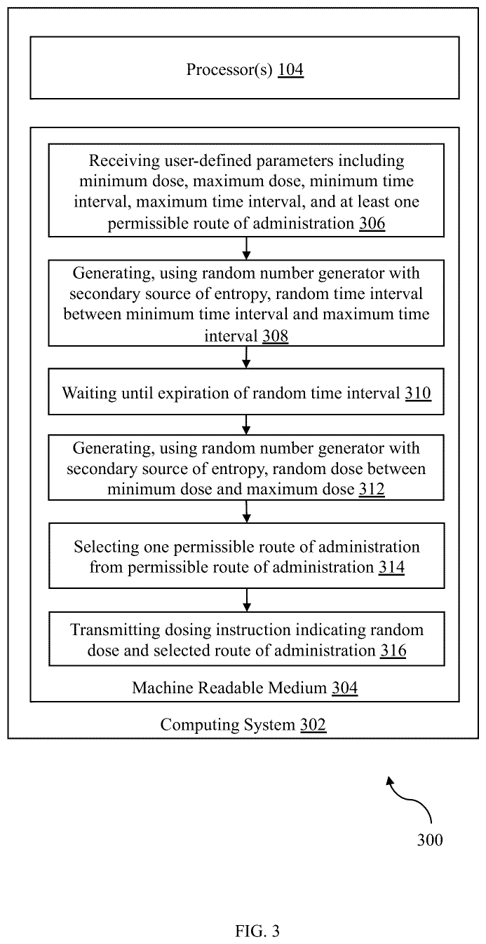

FIG. 3 shows a flow diagram 300 depicting a method for generating and transmitting randomized dosing instructions. At step 302, the system receives user-defined parameters including dose limits, timing intervals, and ingestion method preferences. At step 304, the system generates a random time interval using a random number generator augmented with a secondary entropy source, such as hardware noise or cryptographic entropy. At step 306, the system waits until the selected interval elapses. Once the interval ends, the system proceeds to step 308, where it generates a random dosage and selects a permitted ingestion method. In step 310, the system compiles the instruction using these randomized parameters. In step 312, the instruction is transmitted to the user via pre-selected notification channels. At step 314, the dosing instruction is recorded in a status log including a timestamp, dosage, method, and delivery confirmation. This loop repeats until the user explicitly pauses or stops the application or until a tapering condition is triggered, as shown in step 316.

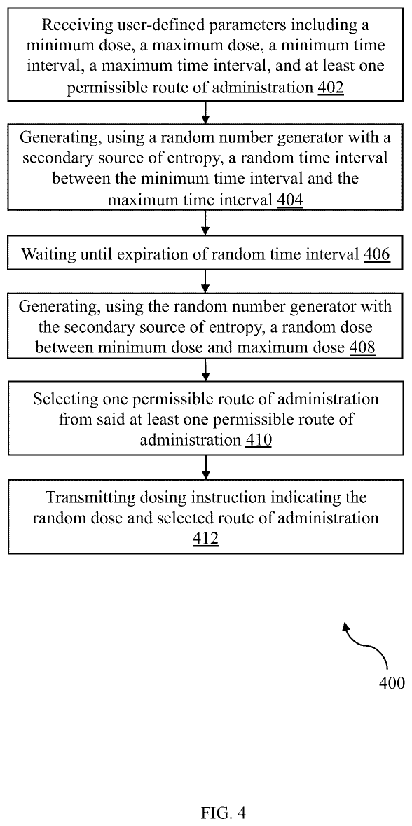

FIG. 4 depicts an exemplary system diagram 400 of a computing environment executing instructions for performing the method. A computing system 402 includes at least one processor 404 and memory 406 storing program instructions for the configuration module 408, randomization engine 410, instruction generator 412, and notification module 414. The memory 406 may also store logs, user preferences, tapering history, and compliance data. The system communicates with external user devices 420 through a communication interface 416 over network 418. The instructions executed by the processor 404 enable the computing system to dynamically generate dosing schedules, randomize parameters, send instructions, and adjust treatment plans based on behavior or tapering logic.

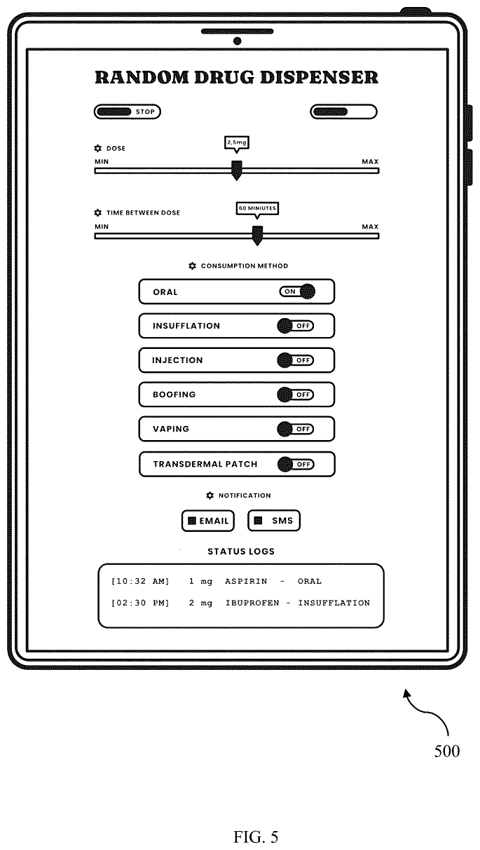

FIG. 5 is an exemplary illustration 500 of a user interface of the Random Drug Dispenser System according to the embodiments of the present disclosure. The user interface includes interactive elements for configuring and managing the dispensing system. A dose configuration slider allows the user to set minimum and maximum dosage levels within permissible limits. A time interval configuration slider is provided to define the minimum and maximum intervals between randomized dosing instructions. Multiple toggle buttons allow users to select and enable permissible ingestion methods, which can include oral ingestion, insufflation, injection, rectal administration (boofing), vaping, and transdermal patch applications. Notification preferences are configurable through options allowing users to choose their preferred delivery channels, such as email or SMS messaging. Additionally, the user interface features clearly labeled control buttons, including a start button to initiate the random dosing schedule and a stop button to cease dispensing instructions. A status log display area is provided within the interface, clearly presenting historical dosing events, including the dosage administered, selected route of administration, and timestamp of each instruction. This log facilitates tracking, compliance monitoring, and user review of the dispensing history.

Deployment of the invention may involve a standalone mobile application for iOS or Android, a desktop application, or a web-based dashboard with secure login. In certain embodiments, the system may be hosted on a preexisting HIPAA-compliant cloud infrastructure to ensure data security, regulatory alignment, and scalable performance in clinical or institutional settings. The cloud backend may provide centralized support for logging, analytics, user management, and clinician oversight, with access controls and encrypted storage to protect sensitive health information. This deployment model enables seamless integration with electronic medical records (EMRs) and external APIs, facilitating interoperability within established healthcare workflows.

In various embodiments, the functionalities described herein can be implemented using a combination of hardware, software, and firmware components operating together in an integrated system. On the software side, the system comprises processor-executable instructions stored on non-transitory computer-readable media such as flash memory, solid-state drives, magnetic storage, or secure cloud-based infrastructure. These instructions, when executed, perform essential operations including user configuration processing, entropy-enhanced random dose and interval selection, dosing instruction generation, event logging, and secure communication with end-user devices. Depending on the deployment context, the software may be implemented as a standalone application, a modular service-based backend, or a distributed system supporting clinician interfaces, user applications, and cloud logging.

On the hardware side, the system may include one or more processors or microcontrollers configured to execute the stored program logic. These processors manage random number generation using cryptographic or hardware-based entropy sources, enforce safety constraints on dosage intervals and amounts, and handle real-time interaction with the user through notification systems. In some embodiments, secure hardware modules may be used to manage sensitive data such as patient configurations, logs, and authentication credentials. The system may also incorporate memory subsystems, both volatile and non-volatile, for managing runtime operations and persistent data storage, including dosing history and compliance metrics.

Memory components may include DRAM or SRAM for fast-access runtime data, and non-volatile memory such as EEPROM or flash storage for longer-term record keeping. The memory may store user preferences, administration method options, tapering settings, and adaptive control logic informed by user compliance. In cloud-enabled embodiments, some or all data may be mirrored or synced with remote storage to facilitate multi-device access, remote clinician supervision, and automated backup.

Communication interfaces may include secure channels for delivering randomized dosing instructions to user devices over Wi-Fi, mobile data (e.g., 4G/5G), or encrypted messaging protocols. Depending on configuration, instructions may be delivered via SMS, push notifications, or encrypted in-app alerts. In cloud-based deployments, the system may leverage HIPAA-compliant hosting environments with enforced encryption standards, access logging, and data residency controls. In some embodiments, the system may also support interoperability with external healthcare systems, such as electronic health records (EHRs), through standard APIs, enabling secure integration into clinical workflows and supporting compliance with data protection and patient privacy requirements.

Scalable implementations may further employ modular architecture wherein each component—such as the configuration manager, entropy engine, instruction dispatcher, and compliance logger—runs as an independent service or function. This design enables updates and enhancements to specific features without disrupting overall system operation. The system may also support over-the-air updates for security patches or logic refinements, allowing clinical parameters, dosing logic, or regulatory configurations to evolve as treatment protocols change.

Overall, the system architecture is designed to provide a secure, reliable, and configurable environment for generating clinically safe but behaviorally unpredictable dosing instructions. Its modular design supports flexible deployment across personal mobile devices, clinician-managed portals, or hybrid cloud-based systems, allowing the invention to scale effectively across individual and institutional use cases.

In all these scenarios, the programming instructions and data structures essential to the system's functionality can reside on various non-transitory computer-readable media or be transmitted in the form of digital or analog signals across diverse physical interfaces. This includes carrier waves, radiofrequency transmissions, optical signals, or electromagnetic energy in other bands. Distributed data storage in multiple locations may enhance redundancy and reliability, ensuring that system operations can continue even if a single node or data center experiences a failure. In alternative embodiments, additional layers of encryption, blockchain-based verification, or secure hardware enclaves could protect the integrity and confidentiality of data exchanged among system components. Overall, embodiments of this disclosure can leverage a versatile mixture of state-of-the-art hardware and software approaches, responding to evolving demands for scalability, fault tolerance, security, and performance while enabling the functionalities described throughout.

These application-level features can be deployed in a variety of formats, including native mobile apps, web-based dashboards, and hybrid models that run in browser windows. In some embodiments, the same functionality is offered through multiple channels, allowing users to control the apparatus from a personal computer, a dedicated in-home display, or even wearable devices. Networking standards such as Wi-Fi, cellular LTE, or newer 5G services can be leveraged to connect the application with cloud servers. Overall, these software capabilities provide flexibility and convenience, ensuring the apparatus seamlessly integrates into modern digital ecosystems and accommodates a wide range of user preferences.

The disclosed invention provides distinct technological and therapeutic benefits compared to traditional drug administration methods. Conventional dosing systems are typically based on fixed schedules, leading to predictable intervals that can foster psychological dependence, anticipatory behaviors, and reduced adherence to therapeutic regimens. In contrast, the present invention leverages controlled randomness through the integration of a randomization engine coupled with a secondary entropy source, delivering unpredictably timed dosing instructions. This technical improvement effectively disrupts conditioned behavioral responses associated with predictable medication schedules, thereby reducing psychological habituation and improving adherence to prescribed treatment protocols. Additionally, the invention facilitates automated tapering strategies by gradually adjusting dose parameters and intervals without direct patient awareness of a scheduled reduction, thus enhancing patient comfort and compliance. The sophisticated computational architecture, including adaptive feedback mechanisms and dynamic logging features, further refines treatment personalization and allows real-time responsiveness to patient adherence data, leading to optimized therapeutic outcomes.

The present invention, although described primarily in the context of substance reduction and medical administration, is adaptable to numerous other therapeutic and non-therapeutic contexts. For example, it can be effectively implemented for chronic pain management by mitigating tolerance development through randomized dosage and timing. Psychiatric or neurological treatments involving medications prone to habituation or tolerance may similarly benefit from randomized dosing schedules, enhancing therapeutic efficacy and reducing the likelihood of dependence. Further alternative embodiments extend the randomized dispensing concept into dietary supplement administration, nutritional management, or other wellness applications where randomized, yet clinically safe administration could improve patient adherence and effectiveness. In clinical research environments, this invention could be employed to rigorously study human behavioral or biological responses under randomized dosing conditions, providing controlled yet unpredictable therapeutic regimens. Additional embodiments might incorporate machine-learning algorithms utilizing historical user interaction and compliance data to dynamically refine randomization parameters, further enhancing patient-specific treatment effectiveness.

Existing computerized medication management solutions rely heavily on deterministic scheduling that, while digitally streamlined, perpetuate predictability and reinforce psychological habituation. Such conventional solutions merely automate routine human processes without addressing the underlying technical problem of predictability in medication scheduling and associated patient behavioral conditioning. These methods inherently lack the technical capability to deliver unpredictable yet clinically appropriate dosage instructions, thereby failing to address the core technological challenge of psychological habituation and anticipatory behaviors induced by predictable timing.

In contrast, the present invention resolves these limitations through the implementation of a novel, technically specialized randomization mechanism. The invention introduces a computational randomization engine integrated with a secondary entropy source, such as cryptographic or hardware-based randomness, thereby providing a sophisticated and genuinely unpredictable mechanism for determining dosage amounts, timing intervals, and ingestion methods. This randomness is precisely bounded within clinically safe parameters, ensuring patient safety while disrupting the psychological predictability characteristic of prior-art medication management systems. The resulting technological advancement significantly improves the capability of medication management systems by effectively eliminating predictable dosing routines, a technical limitation prevalent in conventional computerized dispensing systems.

Furthermore, the invention's comprehensive technical architecture—comprising configuration management, instruction compilation, adaptive logging, feedback-based dosage adjustments, and secure communication modules—represents a tangible improvement in computer-related functionality beyond mere automation of previously known manual processes. The described technology thus fundamentally alters the functionality of computerized medication management systems, offering measurable and clinically significant improvements over conventional scheduling methods by specifically addressing and resolving technical issues related to dosing predictability, patient adherence, and therapeutic effectiveness.

It will be appreciated that while specific implementations of the invention have been described in detail, various modifications, enhancements, and adaptations may be made without departing from the spirit and scope of the invention. Accordingly, the described embodiments should not be construed as limiting but rather as illustrative of the broader capabilities of the invention.

It may be noted that the above-described examples of the present solution are for the purpose of illustration only. Although the solution has been described in conjunction with a specific embodiment thereof, numerous modifications may be possible without materially departing from the teachings and advantages of the subject matter described herein. Other substitutions, modifications, and changes may be made without departing from the spirit of the present solution. All the features disclosed in this specification (including any accompanying claims, abstract, and drawings), and all of the steps of any method or process so disclosed, may be combined in any combination, except combinations where at least some of such features or steps are mutually exclusive.

The terms “include,” “have,” and variations thereof, as used herein, have the same meaning as the term “comprise” or an appropriate variation thereof. Furthermore, the term “based on”, as used herein, means “based at least in part on.” Thus, a feature that is described as based on some stimulus can be based on the stimulus or a combination of stimuli including the stimulus.

The present description has been shown and described with reference to the foregoing examples. It is understood, however, that other forms, details, and examples can be made without departing from the spirit and scope of the present subject matter that is defined in the following claims.

What is claimed is:

1. A method for generating randomized dosing instructions for drug administration, the method comprising:
receiving user-defined parameters including a minimum dose, a maximum dose, a minimum time interval, a maximum time interval, and at least one permissible route of administration;
generating, using a random number generator with a secondary source of entropy, a random time interval between the minimum time interval and the maximum time interval;
waiting until expiration of the random time interval;
generating, using the random number generator with the secondary source of entropy, a random dose between the minimum dose and the maximum dose;
selecting one permissible route of administration from said at least one permissible route of administration; and
transmitting a dosing instruction indicating the random dose and the selected route of administration.

2. The method of claim 1, further comprising automatically adjusting at least one of the minimum dose, the maximum dose, the minimum time interval, or the maximum time interval in successive iterations to reduce overall drug usage according to a tapering protocol.

3. The method of claim 1, wherein generating the random time interval or the random dose comprises acquiring noise data from a hardware-based or cryptographic entropy source to enhance unpredictability.

4. The method of claim 1, wherein the at least one permissible route of administration includes at least two among oral ingestion, insufflation, injection, rectal administration, vaping, and transdermal application.

5. The method of claim 1, further comprising storing each transmitted dosing instruction, including a timestamp, the random dose, and the selected route of administration, in a status log.

6. The method of claim 1, further comprising detecting a user command to discontinue generation of additional dosing instructions and halting transmission of subsequent dosing instructions in response to said user command.

7. The method of claim 1, further comprising transmitting the dosing instruction to a user device via at least one communication channel selected from email, text message, or an in-application notification.

8. The method of claim 1, further comprising receiving user compliance or feedback data and modifying at least one of the minimum dose, the maximum dose, the minimum time interval, or the maximum time interval based on said user compliance or feedback data.

9. A system for generating randomized dosing instructions for drug administration, the system comprising:
at least one processor; and
a memory storing instructions that, when executed by the at least one processor, cause the system to:
receive user-defined parameters including a minimum dose, a maximum dose, a minimum time interval, a maximum time interval, and at least one permissible route of administration;
generate, using a random number generator with a secondary source of entropy, a random time interval between the minimum time interval and the maximum time interval;
upon expiration of the random time interval, generate, using the random number generator with the secondary source of entropy, a random dose between the minimum dose and the maximum dose;
select one permissible route of administration from said at least one permissible route of administration; and
transmit a dosing instruction indicating the random dose and the selected route of administration.

10. The system of claim 9, wherein the memory further stores instructions that, when executed by the at least one processor, cause the system to maintain a status log of transmitted dosing instructions including timestamps, dose amounts, and routes of administration.

11. The system of claim 9, wherein the memory further stores instructions that cause the system to automatically modify at least one of the minimum dose, the maximum dose, the minimum time interval, or the maximum time interval after a predetermined number of dosing instructions in order to implement a tapering strategy.

12. The system of claim 9, further comprising a communication module configured to deliver the dosing instruction to a user device using at least one channel selected from email, text message, or an in-application notification.

13. The system of claim 9, wherein the memory further stores instructions that cause the system to detect and process a user command to discontinue further generation of dosing instructions, and to halt subsequent transmission of dosing instructions in response to said user command.

14. The system of claim 9, wherein the random number generator with a secondary source of entropy includes a hardware-based noise generator or cryptographic library for increased unpredictability of generated intervals and doses.

15. A non-transitory computer-readable medium storing instructions that, when executed by at least one processor, cause the at least one processor to perform a method for generating randomized dosing instructions for drug administration, the method comprising:
receiving user-defined parameters including a minimum dose, a maximum dose, a minimum time interval, a maximum time interval, and at least one permissible route of administration;
generating, using a random number generator with a secondary source of entropy, a random time interval between the minimum time interval and the maximum time interval;
waiting until expiration of the random time interval;
generating, using the random number generator with the secondary source of entropy, a random dose between the minimum dose and the maximum dose;
selecting one permissible route of administration from said at least one permissible route of administration; and
transmitting a dosing instruction specifying the random dose and the selected route of administration.

16. The non-transitory computer-readable medium of claim 15, wherein the instructions further cause the at least one processor to log each transmitted dosing instruction, including at least a timestamp, the random dose, and the selected route of administration.

17. The non-transitory computer-readable medium of claim 15, wherein the instructions further cause the at least one processor to automatically adjust at least one of the minimum dose, the maximum dose, the minimum time interval, or the maximum time interval across multiple iterations to implement a tapering schedule.

18. The non-transitory computer-readable medium of claim 15, wherein the instructions further cause the at least one processor to utilize hardware-based or cryptographic entropy to generate the random time interval and the random dose.

19. The non-transitory computer-readable medium of claim 15, wherein the instructions further cause the at least one processor to receive user compliance data and dynamically modify subsequent random time intervals or random doses based on said user compliance data.

20. The non-transitory computer-readable medium of claim 15, wherein the instructions further cause the at least one processor to cease generating new dosing instructions in response to detecting a user command to discontinue.
